(19) United States
(12) Patent Application Publication
(10) Pub. No.: US 2013/0289570 A1
(43) Pub. Date: Oct. 31, 2013

(54) Tibial Template and Punch System, Tools and Methods for Preparing the Tibia

(71) Applicant: CONFORMIS, INC., Bedford, MA (US)

(72) Inventor: Nam T. Chao, Marlborough, MA (US)

(73) Assignee: CONFORMIS, INC., Bedford, MA (US)

(21) Appl. No.: 13/872,017

(22) Filed: Apr. 26, 2013

Related U.S. Application Data
(60) Provisional application No. 61/639,612, filed on Apr. 27, 2012.

(51) Int. Cl. A61B 17/15

(52) U.S. Cl. CPC A61B 17/157
USPC 606/88

(57) ABSTRACT

Devices, methods and techniques are disclosed that improve a surgeon's ability to assess and optimize the preparation of anatomical support structures in the implantation of knee joint implant components. Described herein are tibial trial apparatuses that provide for preparing a keel cavity in the tibial bone and conduct balancing, rotation, alignment, and/or laxity assessment for implantation of a knee prosthesis. In some embodiments, the tibial trial apparatuses provide for a modular system and can be used in a procedure for cutting, drilling, and keel punching the tibial bone to receive a tibial tray implant.

### RELATED APPLICATIONS

This application claims the benefit of U.S. Provisional Application Ser. No. 61/639,612, entitled “Tibial Template and Punch System, Tools, and Methods for Preparing the Tibia” and filed Apr. 27, 2012, the disclosure of which is incorporated herein by reference in its entirety.

### TECHNICAL FIELD

This disclosure relates to improved and/or patient-adapted (e.g., patient-specific and/or patient-engineered) devices, system and methods for preparing a patient's bones for receiving joint replacement implants and/or assessing the balance, rotation, alignment and/or laxity of the knee. More specifically, disclosed are surgical tools and techniques, including surgical template and punch systems, tools and methods to assist with the preparation of the tibia or similar bones for implantation of a knee joint prosthesis.

### BACKGROUND

When a patient's knee is severely damaged, such as by osteoarthritis, rheumatoid arthritis, or post-traumatic arthritis, it may be desirous to repair and/or replace portions or the entirety of the knee with a total or partial knee replacement implant. Knee replacement surgery is a well-tolerated and highly successful procedure that can help relieve pain and restore function in injured and/or severely diseased knee joints.

In a typical total knee surgery, the surgeon will begin by making an incision through the various skin, fascia, and muscle layers to expose the knee joint and laterally dislocate the patella. The anterior cruciate ligament may be excised and/or the surgeon may choose to leave the posterior cruciate ligament intact—such soft tissue removal often depends on the surgeon's preference and condition(s) of the ACL/PCL. Various surgical techniques are used to remove the arthritic joint surfaces, and the femur and the tibia are prepared and/or resected to accept the component of the artificial implant.

The surgeon may decide to prepare the femur prior to the tibia by conducting a plurality of surgical cuts on the patient's femur, although the order of cuts to the relevant bones is often due to implant system design and/or the surgeon's preference(s). In addition to the femur, the surgeon can prepare the surface of the tibia to accept a combination of tools or templates that desirably verify resected surfaces, alignment, and rotational axis, such as a tibial trial prosthesis or the actual prosthesis on the patient's proximal tibia. The trial or actual prosthesis may include a tibial trial stem that fits the patient's intramedullary canal, a tibial trial or actual metallic tray or plate, or a trial or actual plastic trial insert that fits the tibial tray or plate.

In various techniques, the surgeon can prepare the surface of the tibia by performing one or more cuts on the bone, with the surgeon resecting the articular surface of the bone to receive an implant over the resected surface. The resection can include specific depths of cut(s), posterior slope(s), varus/valgus angle(s), and/or axial alignment(s) that can be unique to every patient. The specific dimensions and/or measurements desirably ensure proper positioning of the artificial joint component assembly, and accurate guiding and cutting of the tibial plateau is typically important to achieve an accurate and appropriate fit of the artificial implant components.

Once the tibia plateau and the femur have been cut, the surgeon may utilize a variety of blocks, spacers, and other tools to ensure proper alignment, rotation, femoral implant thickness, tibial implant thickness, and to make the appropriate cuts or recesses in the bone for receipt of the tibial tray implant. Should the surgeon experience any errors in any of the variables mentioned above, the surgeon may be forced to adjust the resection depth of the femur, the resection depth of the tibia and/or change the tibial tray size with larger or smaller stem size. This can result in longer surgery times, increased frequency of error in implant preparation and/or placement (including rotation and/or alignment errors), and poor surgical outcomes where the knee implant components were not optimally positioned, aligned and/or properly secured/cemented.

Next, the surgeon may select a sizing template and a variety of other tools to determine the correct tibial tray size and positioning. Positioning pin holes may be drilled and filled with pins to hold the template in position. The sizing template may be used to select the keel punch system or used as a base for a keel punch guide system to prepare the canal for the tibial trial stem or the actual tibial tray stem. Additional positioning or securing pin holes may be drilled for use with the keel punch guide system. Typical systems may also require additional keel punch guide systems and tibial sizing templates for each size, tibial tray stem, template or baseplate used. Depending on the number of sizes offered, these types of systems can include a significant number of individual instruments. Once preparation is complete, the template and the keel punch system can be removed and a tibial tray is then placed against the resected bone with an anchor or peg within the intramedullary bore.

There are a variety of alignment tools and templates currently available to assist surgeons in preparing anatomical structures such as bones of a joint, but such systems typically contain too many components and may not be easy to use. Often, a surgeon's attempts to use such systems can lead to malpositioning of implant components, which can significantly contribute to implant component failures and the need for implant revision surgery, prosthetic loosening, arthrofibrosis, deep infection and/or bone loss.

### SUMMARY

According to certain embodiments, a tibial template is disclosed that includes a medial condylar receptacle surface and a lateral condylar receptacle surface. At least a portion of the medial condylar receptacle surface and/or the lateral condylar receptacle surface can have a shape based, at least in part, on patient-specific information. The tibial template can also include a bottom surface and a perimeter. The perimeter can have a shape based, at least in part, on patient-specific information. The tibial template can further include an opening sized and shaped to accommodate a cutting tool, a drilling tool, and/or a keel punch.

According to certain additional embodiments, a system for treating a knee joint of a patient is disclosed. The system can include, at least, a femoral implant and a tibial template. The tibial template can include a medial condylar receptacle surface and a lateral condylar receptacle surface. At least a portion of the medial condylar receptacle surface and/or the lateral condylar receptacle surface can have a shape based, at least in part, on patient-specific information. The tibial template can also include a bottom surface and a perimeter. The perimeter can have a shape based, at least in part, on patient-specific information. The tibial template can further include an opening sized and shaped to accommodate a cutting tool, a drilling tool, and/or a keel punch.

### BRIEF DESCRIPTION OF THE SEVERAL VIEWS OF THE DRAWINGS

FIG. 1 depicts a side view of a template constructed in accordance with various embodiments;

FIG. 2 depicts an anterior view of the tibial template of FIG. 1;

FIG. 3 depicts a bottom view of the tibial template of FIG. 1;

FIG. 4 depicts a top plan view of the tibial template of FIG. 1;

FIG. 5 depicts an isometric view of the tibial template of FIG. 1;

FIG. 6 depicts a top plan view of a tibial template bushing;

FIG. 7 depicts a bottom view of the tibial template bushing of FIG. 6;

FIG. 8 depicts a side view of the tibial template bushing of FIG. 6;

FIG. 9 depicts a side view of one alternative embodiment of a tibial template bushing;

FIG. 10 depicts an isometric view of the tibial template bushing of FIG. 9;

FIGS. 11A and 11B depict isometric exploded views of a tibial template and associated tibial template bushing;

FIGS. 12A and 12B depict isometric views of the tibial template and tibial template bushing assembly of FIGS. 11A and 11B in an assembled state;

FIG. 13 depicts a top plan view of the tibial template and tibial template bushing assembly;

FIGS. 14A and 14B depicts bottom views of the tibial template and tibial template bushing assembly in open and locked positions;

FIGS. 15A and 15B depict an anterior side view and a standard side view of the tibial template and tibial template bushing assembly;

FIG. 16A depicts a resected femur, a resected tibia and a femoral block to verify planar cuts;

FIGS. 16B and 16C depicts a side view of the femurs with resected cuts and the proposed profile of the femoral implant and/or femoral trial;

FIG. 17 depicts a resected femur with an associated trial femoral implant, a tibia and one embodiment of the tibial template that assists with balancing, alignment and rotation of the implant components;

FIG. 18 depicts an isometric view of a tibial template and associated tibial surface, highlighting positioning pin holes;

FIG. 19 depicts a top plan view of the tibial template and associated tibial surface FIG. 18;

FIG. 20 depicts a top view the tibial template and associated tibial surface of FIG. 18, including a tibial template bushing and highlighting a bore through to the tibial intramedullary canal;

FIGS. 21A and 21B depict posterior and anterior views of the tibial template and associated tibial template bushing assembly;

FIG. 22 depicts a top plan view of the tibial template, highlighting the bore through the intramedullary canal and the keel punched extensions; and

FIG. 23 depicts a top plan view of the resected tibial surface, highlighting the bore through the intramedullary canal and the keel punched extensions.

### DETAILED DESCRIPTION

The present disclosure describes improved tools, systems and methods for aligning and preparing anatomical support structures for placement of joint replacement/resurfacing implant components. Various embodiments include improved patient-specific or patient engineered tibial template, alignment and keel punch apparatus (hereinafter “tibial template”) and associated methods that desirably overcome and/or address various disadvantages of existing systems. The various embodiments described herein may be used to facilitate total knee surgery, bicompartmental knee surgery or unicompartmental knee surgery. In addition, the various features described herein may be used during cruciate retaining surgeries or non-cruciate retaining surgeries.

Various embodiments described herein may include patient-specific or patient engineered features for each surgical patient, with each tibial template tailored to an individual patient's joint morphology. In at least some embodiments, the system may be designed as an assembly that comprises a patient specific tibial template with an integrated keel punch system and modular keel drill guide.

In various embodiments, each piece of the tibial template assembly can be uniquely tailored to an individual patient's anatomy, which may utilize images taken from the subject. The manufacturer can design the patient-specific tibial template assembly using the joint image from a patient or subject, wherein the image may include both normal cartilage or bone or diseased cartilage or bone; reconstructing dimensions of the diseased cartilage or bone surface to correspond to normal cartilage or bone (using, for example, a computer system); and designing the tibial template to exactly or substantially match the perimeter dimensions of the tibial resected surface, the normal cartilage surface, a healthy cartilage surface, a subchondral bone surface, and/or various combinations thereof (including height, width, length, medial/lateral, and posterior/anterior angles). The image can be, for example, an intraoperative image including a surface and/or feature detection method using any techniques known in the art, e.g., mechanical, optical, ultrasound, and known devices such as MRI, CT, ultrasound, and other image techniques known in the art. The images can be 2D or 3D or combination thereof to specifically design the tibial template assembly.

In various embodiments, the template may comprise patient-specific, patient-engineered and/or standard sized femoral condyle surfaces and/or “receptacles” (or various combinations thereof). These receptacles can incorporate varying posterior/anterior angles, varus/valgus angles, and/or other varying dimensions. Each template can be designed to match a patient-specific knee implant prosthesis, insert and/or trial knee prosthesis component. Optionally, different sizes may be made available (e.g., differing thicknesses, differing thickness combinations and/or varying outer dimensions) enabling the surgeon to make adjustments to the resected femur and/or tibia, as well as to determine whether a proposed alteration might positively and/or negatively affect the knee's performance.

In various embodiments, the template may include an integrated or modular drill guide. In some embodiments, the drill guide may be modular and have a quick connect mechanism for connection to the template when the surgeon is prepared to drill and insert the tibial tray. The drill guide may be sized to accommodate a “one-size fits all” drill reamer or other drill sizes, or the drill guide may be designed to several standard sizes for the surgeon to use. The drill guide may be integrated into the template to provide more of a positive stop for the surgeon when using the drill.

In some embodiments, the template may include an integrated or modular keel punch system. In at least one preferred embodiment, a keel punch system can be integrated into the template to match standard size tools and tools provided to the surgeon. If desired, the keel punch system may be modular and attached to the template when the surgeon has sized the tibial tray and is ready to insert the tray into the tibia. The keel punch system may be manufactured as patient-specific, patient engineered and/or made available with standard sizes.

In various embodiments, a multi-purpose tibial template can be useful in total knee surgery in conjunction with either conventional tibial and femoral alignment or spacer guides.

Various devices, systems and methods suitable for improving the preparation and placement of knee replacement implant components are disclosed herein. In various embodiments, the systems can include a femoral trial assembly and tibial template apparatus, with the surgeon employing various methods to (1) determine a desired location of the tibial prosthesis component on the patient's tibia, (2) determine the position of the tibial tray stem and keel of the tibial prosthesis component, (3) adjust the resection depth of the tibia if necessary, and (4) verify and/or accommodate alignment, laxity and/or rotation of the tibia relative to the femoral implant component.

Various features of some embodiments disclosed herein may be especially useful to surgeons during knee replacement/resurfacing surgery, as the correct sizing and placement of partial or total knee prosthesis components during surgery will desirably replicate or approximate the normal wear characteristics of a specific knee.

The normal knee joint typically combines a full range of flexion and extension, and a few degrees of laxity in rotation, with great strength and stability. The unique curvature of the femoral and tibial joint surfaces, combined with the manner of placement and attachment of the ligaments, desirably allows the knee joint to remain stable through all positions. Typically, knee joint stability is provided primarily by two pairs of ligaments, the cruciates and collaterals, with supplementary help from the joint capsule, motion-controlling muscles and other soft tissues. The anterior and posterior cruciate ligaments are strong short ligaments in the center of the knee that prevent abnormal anteroposterior joint displacement. The medial and lateral collateral ligaments are located outside the knee joint itself. They may be considered stress-resisting expansions of the joint capsule. They extend from their attachments above the joint to the tibia and fibula below and provide side-to-side stability.

In various embodiments, the combination of the various ligaments and the prosthetic implant components should cooperate to create a normal knee function that allows smooth, uninterrupted motion along the normally lubricated, low-friction articular surfaces. Many factors are interdependent in this motion, and a failure of one component or factor can eventually lead to a breakdown of the others as well as a catastrophic failure of the entire joint in certain circumstances. Desirably, the various embodiments described herein can facilitate proper bone preparation for receiving one or more knee replacement/resurfacing components, including the use of preoperative planning templates (e.g., tibial templates) for determining the correct size, position, laxity, alignment and rotation of the knee (and the associated proper placement of implant components to accomplish proper knee motion), resulting in a successful surgical procedure.

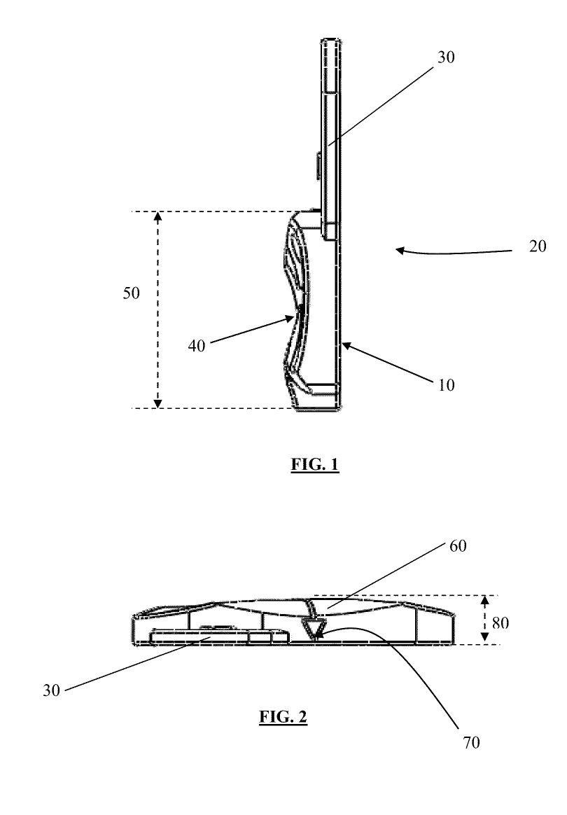

FIG. 1 depicts a side view of a tibial template 20, showing a user handle 30, a flat bottom surface 10, a pair of patient-specific condylar medial and lateral receptacles 40, and a patient specific width 50 of the tibial template. The user handle 30 can be designed with a unique ergonomic shape that makes it easier for the surgeon to handle. It can include various specified lengths to provide easy maneuverability and grasping surface areas during a surgery. Also, the user handle 30 desirably includes smooth or atraumatic edges to ensure that the user does not get injured or cut on a corner. The user handle 30 may be any design shape and length that accommodates the surgeon's manipulation of the device during open knee surgery, e.g., it may be designed to have a grip bar, have the ability to have an extension attached and/or incorporate a friction-reducing surface to accommodate fluids such as blood that may be deposited on the handle during the surgical procedure. In various embodiments, depending on the type of surgery that is conducted, the handle may be designed offset or in the center of the template.

The flat bottom surface 10 can include a perimeter and/or edge shape that incorporates various patient specific features. For example, the patient images obtained can be processed to identify the perimeter shape of the tibia at one or more desired cut levels and/or along desired cut planes of the bone structure, and the tibial template bottom (or other portions of the template, if desired) can be shaped to substantially match some or all of the perimeter of the resected tibia. In various other embodiments, anatomical data from the processed images can be used to model and/or replicate one or more patient specific features of the condylar receptacles 40. These receptacles may be patient specific on either or both of the medial and/or lateral sides, as desired. The patient specific nature of the dimensions may vary. The manufacturer may choose to make the medial or lateral sides patient specific. The anterior/posterior dimensions, the radius, and the medial/lateral dimensions may be standard patient sizes obtained from a general database or using patient specific derived dimensions, such as the width 50 of the tibial template. The tibial template dimensions will desirably significantly assist the surgeon during his or her preoperative planning and templating of the patient's anatomy (i.e., using tools such as the tibial template) for determining the correct size, position, laxity, alignment and rotation in which to place the prosthesis. The tibial templates may be offered in several sizes, and they may be employed to measure and/or correct any discrepancies in the laxity, alignment, and rotation of the knee joint when the surgeon conducts their laxity or balancing tests for the patient during flexion and extension. Depending upon the outcome of such tests, the surgeon may choose to increase or decrease the size, position and/or dimensions of implant components and/or surgical cut planes on the bone. If desired, the surgeon may choose to select another tibial template, provided by the manufacturer in a different size or shape than the previous template used by the surgeon, which can then be utilized to determine if the new size/shape can successfully reproduce the smooth, gliding, and uninterrupted motion of the knee implant. If so, it may be desirous for the surgeon to choose to increase or decrease the size, position and/or dimensions of implant components and/or adjust the surgical cut planes on the bone to replicate the new motion (reflected by the new template). This process may be repeated as necessary.

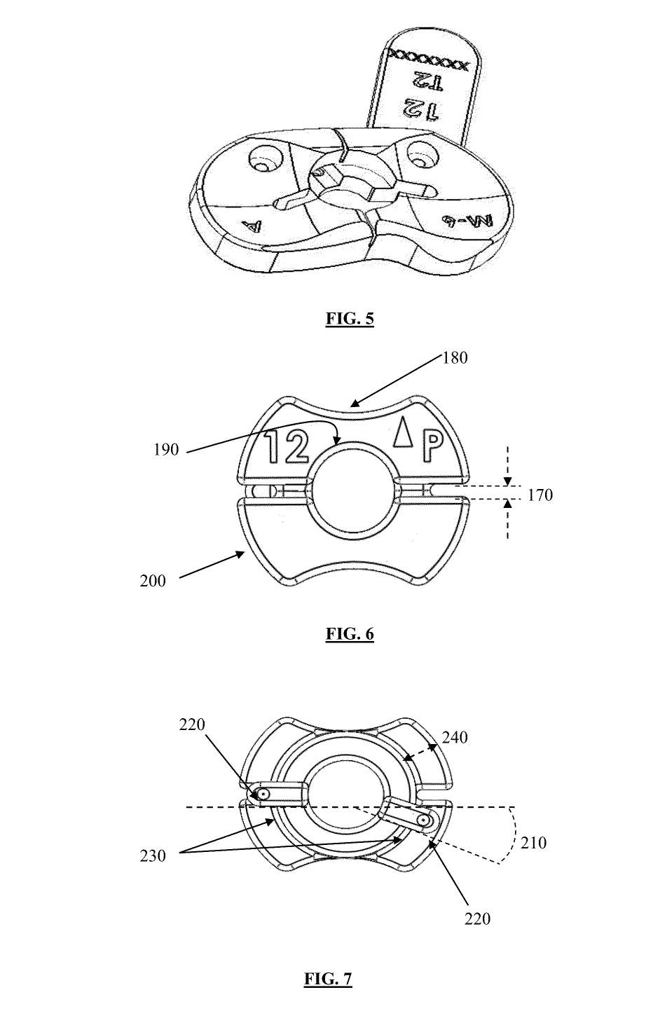

FIG. 2 depicts an anterior view of the tibial template 20, showing an anterior relief surface 60, a bushing indicator 70, the template height 80, and a user handle 30 of the tibial template. The anterior relief in this embodiment is designed as a sloped chamfer, which desirably presents an angled face towards any soft tissues that may impinge against the relief surface, and which may serve to decrease tissue inflammation in the event of unwanted or excessive soft tissue contact. One or more relief surfaces may be incorporated into the template as desired, and in various embodiments various relief surfaces may be positioned adjacent significant soft tissue structures and/or attachment points, which could include any adjacent ligamentous structures such as the LCL, the MCL, the ACL and/or the PCL. A bushing indicator 70 is positioned adjacent to the anterior relief surface, which in this embodiment provides a visual representation for the surgeon to align the bushing (see FIG. 6) onto the tibial template prior to locking the bushing in place. In the current alignment, the indicator comprises a thin gutter, but this indicator could be designed in a wide variety of ways. The indicator may be a mechanical indicator or it may be a raised surface to show the surgeon how or where to place the bushing.

The height 80 of the tibial guide template may be patient specific, may be patient-derived and/or may be derived from a standard patient database. If the tibial guide template height 80 is patient specific, the image data obtained from the patient can be analyzed to manufacture a desired and/or proper height to estimate a thickness to ensure that the knee prosthesis functions properly, which in various embodiments may be a manner similar to a normal knee and/or similar to a corrected and/or optimized knee anatomy. The height can be useful in determining the proper and/or optimal laxity, alignment and/or desired rotation of the knee for a desired surgical outcome. In various embodiments, an optimal template height (and/or other anatomical features) can be derived from patient anatomical data, and then an additional series of template heights (and/or additional series of anatomical features) can be derived and manufactured that “bracket” or bound the optimal value, with the additional templates used during the surgical procedure to test the knee joint during the surgical procedure to optimize the surgical repair. For example, if patient-specific data indicates a template height of 8 mm is the optimal height for the template, then additional templates having heights of 4 mm, 6 mm, 10 mm and 12 mm may be created. During the surgical procedure, it may become apparent that the 8 mm height appears too high, which could result in “over-stuffing” of the joint if the corresponding implant components were utilized, but the use of the 6 mm or 4 mm template may indicate more appropriate implant components (or alternatively indicate additional bone resection is appropriate, or the resulting test may alternatively indicate that the 8 mm selection remains the most appropriate choice).

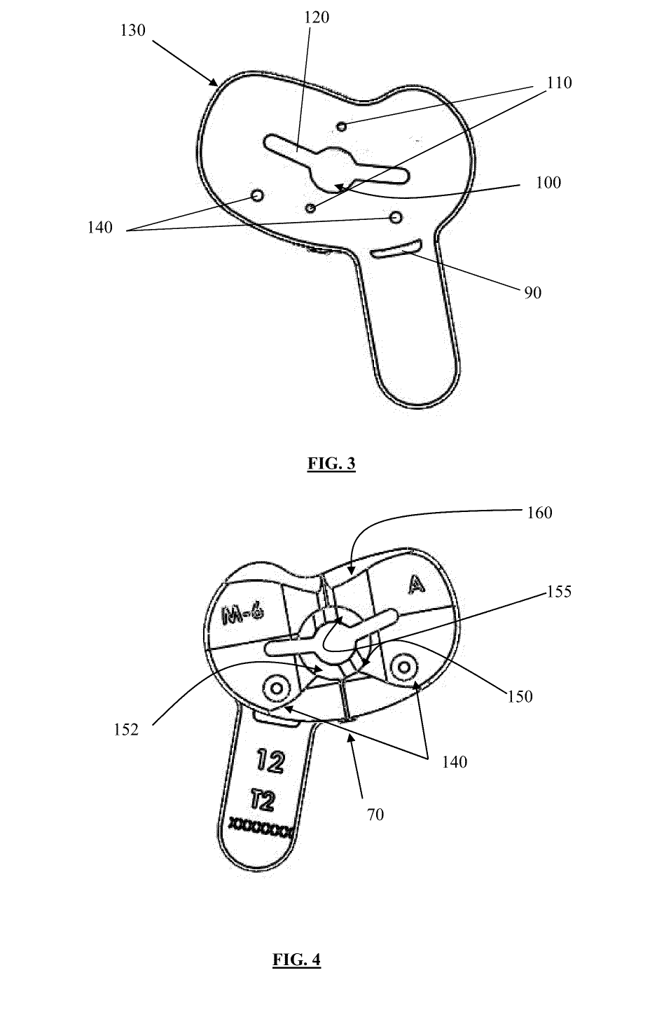

FIG. 3 depicts a bottom view of the tibial template, showing an alignment or edge window 90, a pair of position pin holes 140, a pair of bushing detent holes 110, and a keel guide 120 which includes a drill guide hole 100. Also can be seen is the outer perimeter 130 of the tibial template, which in this embodiment is formed in an approximate shape of the tibial surface after the tibial bone has been cut by the surgeon. The edge window 90 is designed to facilitate the surgeon's visualization of the peripheral anterior edge of the resected tibia. This window can assist with guiding and/or confirming the surgeon's placement of the tibial template in the proper location without placing it too posterior. The shape of this window may vary to give the surgeon the best view of the peripheral edge of the tibia. The shape dimensions may have a wider window, or may be a variety of other shapes (e.g., semi-circles, arcs, triangles, squares, and circles). While in this embodiment the window is formed generally perpendicular to the upper or lower surface of the template, other alternative shapes, including angled shapes and/or orientations may be used, to accommodate the use of the tool in less-invasive and/or minimally invasive surgeries (where it may be difficult and/or impossible for the surgeon to “look down” through a vertically-oriented opening).

In the embodiment shown, the position pin holes 140 are located proximate the medial and lateral sides of the condylar receptacles. These position holes can include a counterbore or other depth limiting feature that desirably facilitates the guiding of the drill once a satisfactory position of the tibial template has been established. The counterbore of the position pin holes prevents the drill from exceeding the proper depth for drilling. Once drilling and preparation are completed, positioning pins may be placed within the holes to secure the template to the underlying bone and prevent or limit unwanted or excessive movement.

The bushing detent holes 140 may also be seen in the bottom view of the tibial template. The detent holes can be used to lock or secure the bushing in place within the template and may also provide an audible signal (if desired) to the surgeon that the bushing has been adequately secured. Other alternative designs may include a variety of other locking mechanisms to secure the bushing into place. Mechanisms such as tabs, set screws, rails, dovetails, and equivalent mechanisms may be used.

The keel guide 120 and the drill guide 100 can be used when the surgeon has determined a desired proper alignment, laxity, rotation, and/or stability of the knee, to begin the proper placement of the tibial prosthesis. The bushing (see FIG. 6) is designed to be inserted into the drill guide hole 100, and the keel punch is designed to be inserted into both the drill guide hole 100 and the keel guide 120 to make the proper holes and cut-outs to fit the stem of the tibial tray. In this embodiment, it places the keel guide and the drill guide hole in the center of the tibial template, however, in various alternative embodiments these holes/cut-outs may be placed in offset positions and/or at various keel angles to accommodate a desired location and/or construction/strength of the stem and keel, and their placement within the intramedullary canal of the tibia (or other bone location).

FIG. 4 depicts a top view of the tibial template, highlighting a posterior relief 160, a bushing counterbore 150, the position pin holes 140 and the bushing indicator 70. The posterior relief in this embodiment is designed as a sloped chamfer to decrease tissue inflammation and/or impingement where the ligament may contact the surface of the template. The bushing counter bore 150 is designed into the template to provide a positive stop for the bushing when inserted into the center of the tibial template, and it can have a channel for the bushing legs (see FIG. 7) to seat into. The channel 152 is used to secure the bushing legs by “sandwiching” or trapping the legs and preventing excessive movement during drilling. The channel has a wall 155 within to prevent over rotation of the bushing while turning it clockwise. The channel wall 155 can desirably allow only a 90 degree rotation of the bushing until the bushing legs hit the wall. There are many other alternative embodiments that can be used to secure the bushing into the tibial template for quick connect and release; these can include two 180 degree channels that are placed within the counter bore to help align the bushing (i.e. the bushing has external dovetails or rails that fit like “lock & key” type mechanisms), the bushing may also be secured by set screws, or may be threaded, and many combinations thereof.

FIG. 6 depicts a top view of the tibial template bushing 200. The bushing can include ergonomic features 180 that allow the surgeon to easily grasp the bushing. The bushing may also have other material coatings (i.e. rubber or other friction type surface) that are formed or coated onto the bushing to ensure that the bushing 200 minimizes and/or eliminates slippage. The bushing includes a drill guide 190 that is designed to fit standard drills or reamers that are commonly available in surgical operating rooms. However, the manufacturer may produce or customize a drill that will fit within the drill guide 190. The tibial template bushing also has an alignment indicator 170 that matches the tibial template bushing indicator 70. The alignment indicator 170 on the bushing currently is designed as a set width for the surgeon to easily visualize the indicator and ensure it is locked when it matches with the bushing indicator 70. The indicator is one exemplary embodiment, may be designed as the various indicators known in the art.

FIG. 7 depicts a bottom view of the tibial template bushing. The bottom view shows the bushing legs 220 (and keel angles 210 and the bushing detents 230) that fit within the tibial template keel guide 120. The bushing legs 220 can be designed to have a specified length to fit within the channel of the tibial template. Since the bushing legs in this embodiment are designed to ensure that the legs are “trapped” within the channel 152, the legs will desirably be of sufficient length to withstand moderate rotational force and/or tensile force. Also, the legs are designed to match the keel angles 210 on the tibial template keel guide 120.

The bushing legs 230 may also have detents integrated within the legs to facilitate locking of the bushing to the tibial template. These detents will fit within the detent holes of the tibial template 110 to provide an audible sound, if desired. The bushing may also have a ledge 240 to allow for a surgeon's fingers to be placed beneath the ledge for lifting or rotational purposes.

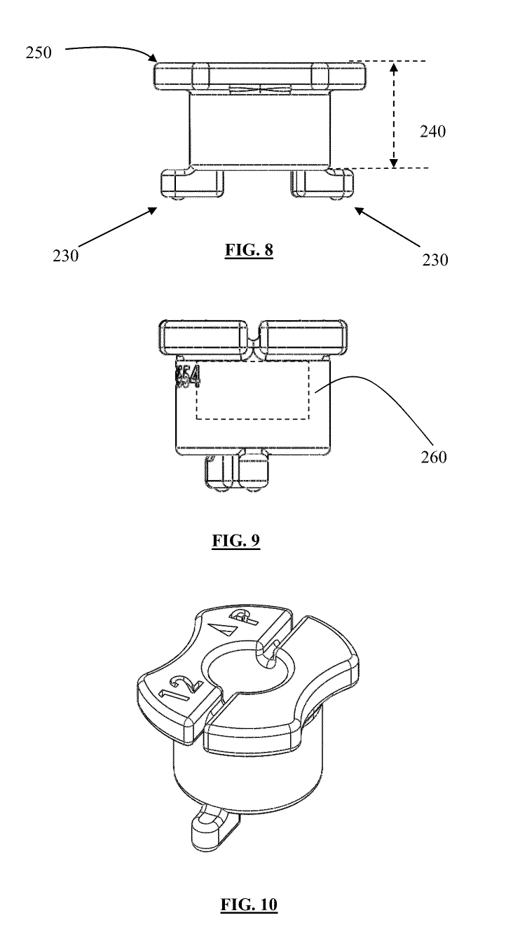

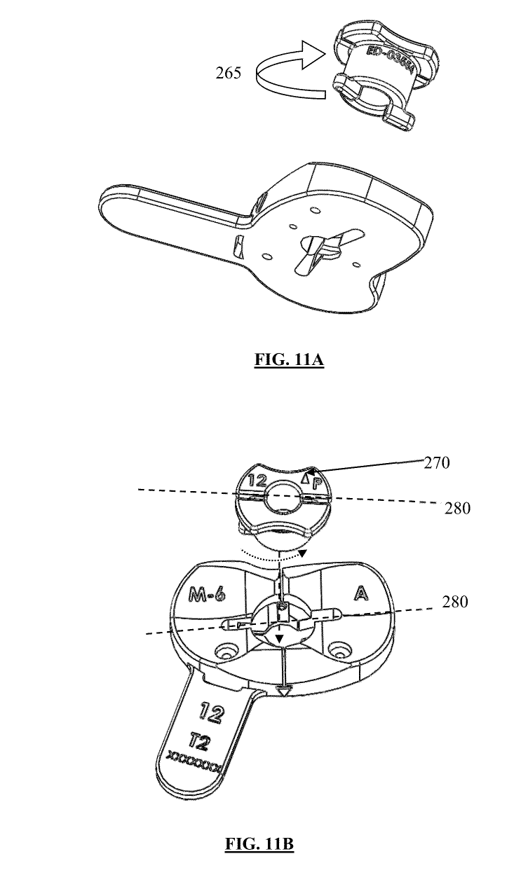

FIG. 8 depicts a side view of a preferred embodiment of a bushing for use with a template. The height 240 of the bushing can be designed to control the depth of the drill into the intramedullary canal of the tibia. The height will desirably act as a positive stop for the drill so the surgeon does not have to subjectively gauge the depth of drill penetration and possibly drill too shallow and/or deep. The surgeon may use alternative embodiments of the bushing as a positive stop as depicted in FIG. 9. This alternative embodiment inserts a counter bore into the bushing so the drill may also have a positive stop. This alternative design may allow the bushing height 240 to be smaller, yet accomplish similar objectives. As depicted, the bushing has smoothed or atraumatic edges 250. The atraumatic edges can include beveled, radiused or chamfered edges to prevent sharp edges and/or points of the device from causing injury and/or pierce protective clothing, such as latex surgical gloves.

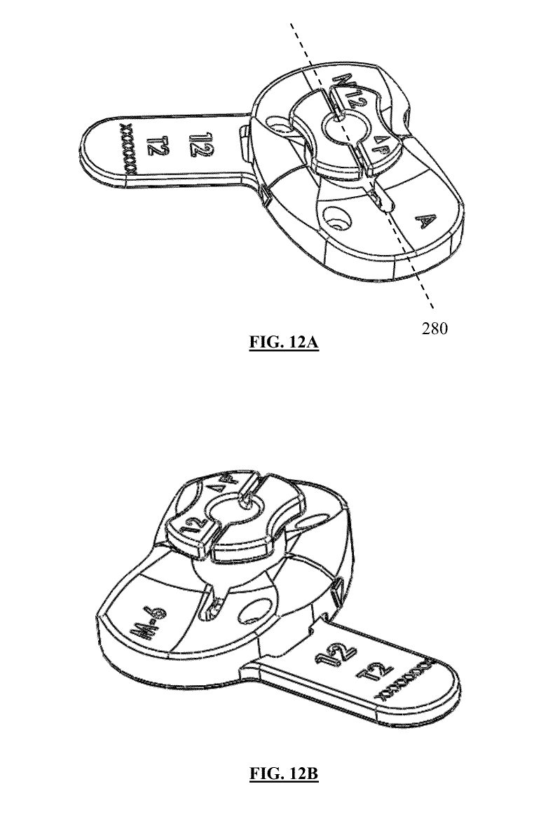

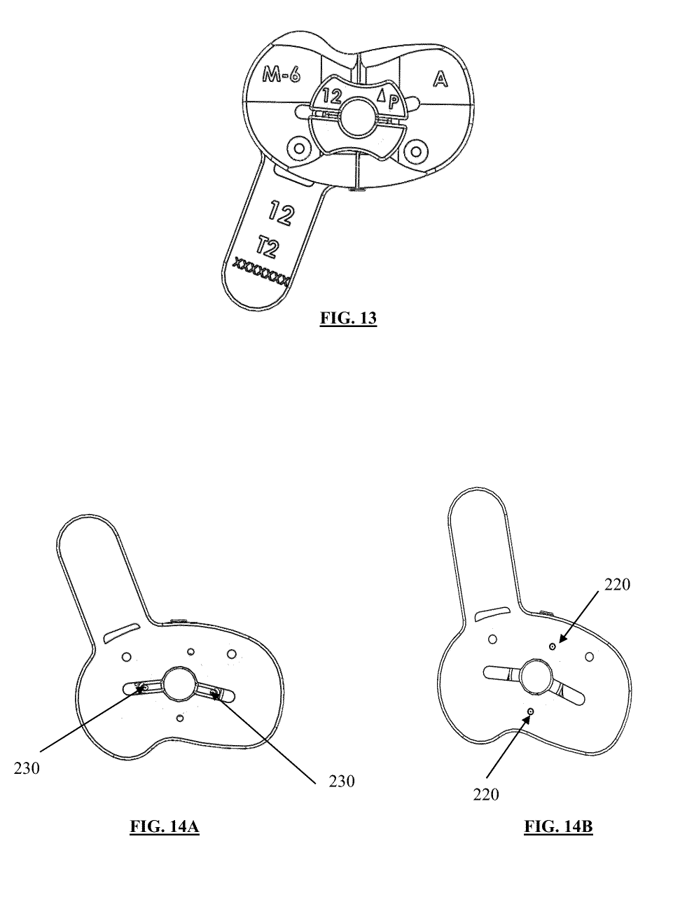

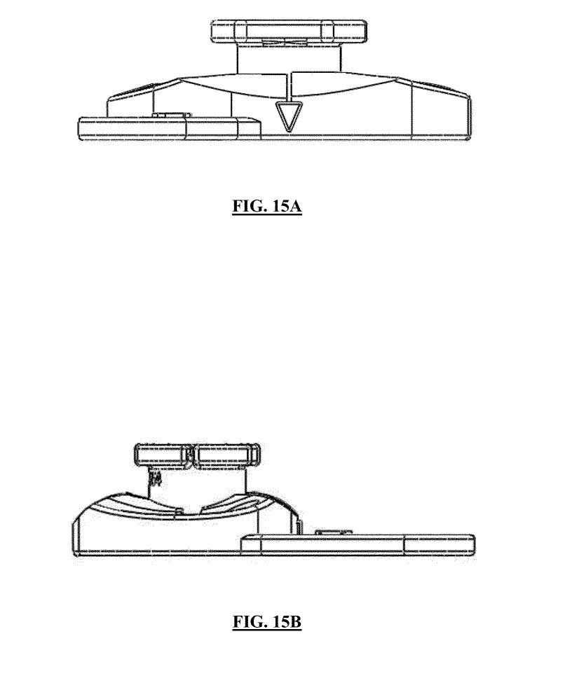

FIGS. 11A and 11B depict an isometric exploded view of the tibial template and the tibial template bushing ready for assembly. FIG. 11A shows an exemplary 90 degree rotation 265 to match the bushing legs 230 with the keel guide 120. Once the bushing legs 230 are matched with the keel guide 120, the bushing will easily sit in the channel and provide a positive stop to the surgeon. FIG. 11B shows the proper alignment 280 of the bushing legs 230 with the keel guide 120. In various embodiments, the manufacture may design other textual indicators 270 to help the surgeon understand how to place and align the bushing into the tibial template, as well as identify the appropriate bushing (if multiple sizes and/or shapes are provided) and drill combination. This embodiment shows that the posterior “P” should be facing the posterior side of the tibia. FIGS. 12A and 12B depict isometric views of the tibial template and the tibial template bushing assembly with the bushing legs 230 aligned 280 with the tibial template keel guide 120. FIG. 13 depicts a top view of the tibial template and tibial template bushing assembly—another perspective of the tibial template and bushing aligned prior to its locked position.

FIGS. 14A and 14B depict bottom views of the tibial template and tibial template bushing assembly in its open and locked positions, respectively. FIGS. 12A, 12B and 13 show the bushing in an unlocked position. When the tibial template and bushing assembly are viewed from the bottom, it is easier to visualize whether the bushing is unlocked or locked. When the bushing is unlocked, the bushing legs 230 will desirably align directly with the tibial template keel guide 120. However, FIG. 14B shows the bushing in a locked position, where the bushing legs are no longer aligned with the tibial template keel guide 120, and are now sitting in the detent holes 220. The bushing in the locked position will desirably prevent excessive component movement during drilling.

Procedure

Total knee surgery is a challenging intervention that helps patients suffering from knee osteoarthritis or other injury or disease improve their range of motion, reduce their pain with daily activities, and help them become more active. Traditionally, knee surgery is performed with bone cuts as the main focus of the procedure with less attention paid to the balancing, alignment and rotational adjustment to the knee, even though balancing, alignment and rotational adjustments have been shown to be important to the physiologic functioning of the knee joint. The four major ligaments (MCL, LCL, ACL and PCL) form the static stabilizers of the joint while muscle-tendon structures at the knee provide dynamic stability. The complex balance between these two stabilizing systems is important to the successful outcome of cruciate retaining knee surgeries. This is also true even during cruciate sacrificing surgeries that may only depend on the MCL and the LCL because the ACL and/or the PCL were sacrificed for conventional knee surgeries.

Currently, balancing, alignment and rotational adjustment is assessed in a very subjective manner. After implantation of trial components, the surgeon visually inspects the range of motion, tracking of the patella and manually tests medial/lateral/posterior/anterior knee stability by applying stress in the appropriate directions, and testing the laxity of the collateral or other remaining ligaments under flexion and extension. One standard approach is generally to determine the stability of the knee at two limb orientations: at full flexion and full extension. Once stability has been assessed, if changes are desired the surgeon can attempt to adjust the balancing, alignment, and rotation of the knee by making appropriate changes—re-cutting bone, releasing soft tissues, choosing other implant sizes, and/or otherwise repositioning and/or reorienting the various implant components. Unfortunately, true assessment of the balancing, rotation, laxity and alignment of the knee throughout the entirety of the range of knee motion may not be accomplished until the end of the surgery, when the femoral trial, tibial tray and tibial trial inserts are secured and cemented into place, at which time further alterations to the implant may be difficult, impossible and/or not feasible.

In an effort to address this deficiency, the various device and methods described herein, including the use of the described tibial templates, may facilitate the intra-operative assessment of laxity, balancing, rotation and alignment of the knee throughout an entirety of the range of motion. The laxity, balancing, rotation and alignment of the knee can be relative to the gap in the knee, so the surgeon can assess many of these items by considering an optimal gap in the knee joint components. The tibial template, femoral trial and associated system will desirably offer a design to assess the balancing, laxity, rotation and alignment of the knee to accurately choose the right implant size, to choose the proper positioning of the tibial implant, and to reduce poor implanting techniques early in the procedure. In order to achieve these objectives, it is important to also utilize appropriate surgical techniques to cut accurate femoral and tibial surfaces at ideal levels and angles to use the improved tibial template.

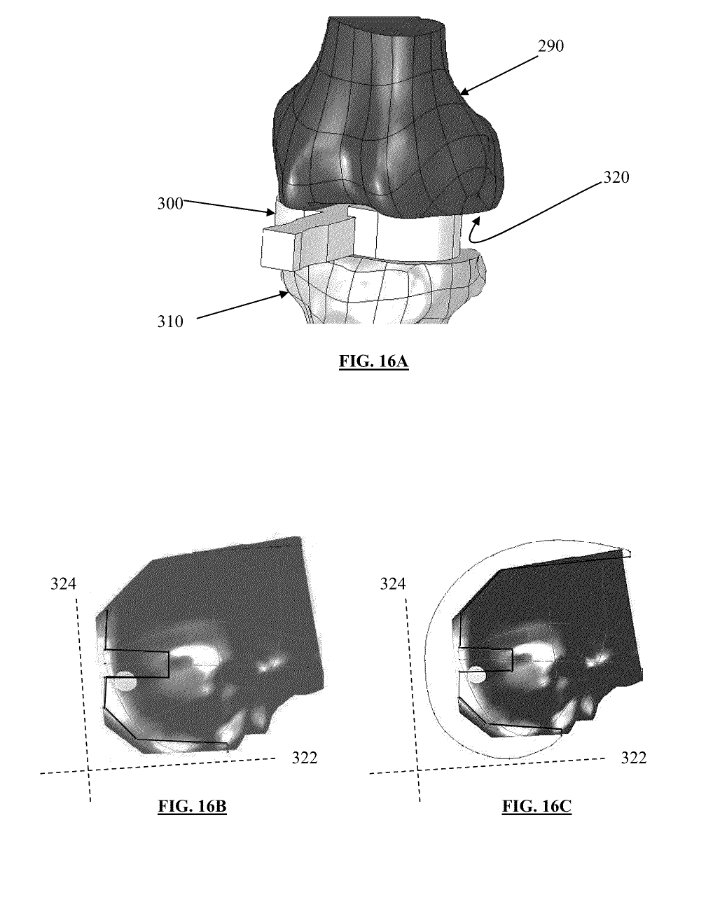

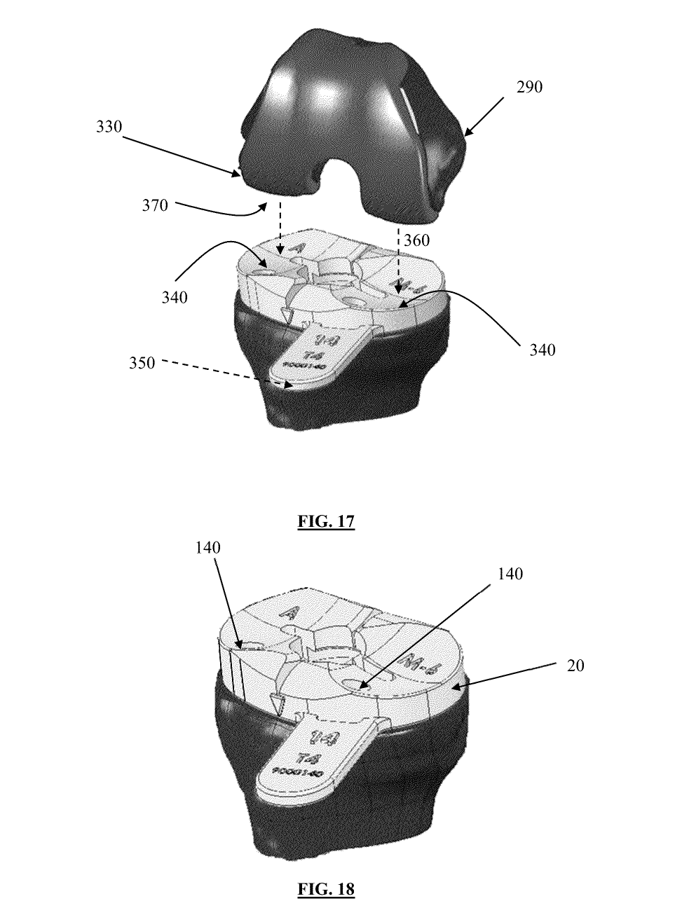

FIG. 16A depicts a resected femur 290, a resected tibia 310 and femoral blocks 300 to verify planar cuts 320 and potential height of knee prosthesis, and assess knee balancing in extension and flexion. This conventional method of assessing knee balancing typically requires a significant amount of time to accomplish and, because it generally measures soft tissue tension at a single point in the knee motion (i.e., only at a certain angle in flexion and/or tension), it may not immediately identify where a subsequent resectioning of the tibia or femur to accommodate improper balancing and laxity in one alignment (e.g., flexion) may have undesirable effects on the soft tissues and/or knee performance in another alignment (e.g., extension). Thus, the conventional system requires that the surgeon repeatedly insert and remove spacer blocks and conduct a certain number of “back and forth” assessments (between flexion and extension) before accomplishing a given resection and/or other surgical correct, to ensure that changes do not unacceptably affect the knee in various alignments. Conventional assessments in extension-flexion balancing at 0° and 90° may entail: assessing knee motion, making a minimum tibial resection, assessing knee motion, resecting portions of the posterior femur to the same thickness as the components, assessing knee motion, making fine cuts or other adjustments to components, and finally finishing at extension. The process may be further complicated by additional adjustments to resect the tibia and femur when attempting to prepare the tibia to receive the tibial tray implant.

FIG. 16B shows an exemplary side view of a traditional assessment for balancing, rotation, alignment and laxity of the knee prosthesis. Multiple femoral blocks and spacers can be used in flexion 322 and in extension 324 to make this assessment. The flat resected surfaces of the femoral and the tibia cut surfaces do not reflect the true profile of a patients' knee, which can lead to inaccurate balancing, rotation, laxity and/or alignment of the actual knee prosthesis. Assessment using femoral blocks and spacers can lead to multiple unnecessary steps in the surgical process, excessive cuts of the tibia or femur, improper balancing and rotational alignment of the knee, and remedial measures for improper fixation or positioning of the tibial tray. In addition, if the knee is not properly balanced, aligned, rotated, or properly tensed, the knee may feel too tight, too loose or unstable. Instability is a significant factor in re-operations, to correct a malfunctioning, unstable and/or painful knee replacement.

FIG. 16C is a side view of a resected femur, with the profile of a patient-specific femoral trial implant and/or an actual patient specific femoral implant superimposed. When such an implant is placed on the femur, its interaction with embodiments of a tibial template can predict the actual performance of the knee prosthesis throughout its entire range of motion—including assessments of balancing, rotation, laxity and/or alignment of the actual knee prosthesis. Placing the femoral trial onto the femur, and the template on the tibia, and moving the knee during the assessment of the balancing, rotation, laxity and alignment of the knee accurately mimics the true range of motion (i.e. the profile and depth) of knee implant prosthesis during flexion 322 and extension 324.

FIG. 17 depicts a resected femoral bone 290 with an attached femoral trial implant 330, a resected tibial bone and one embodiment of the tibial template to assist with balancing, alignment and rotation of the implant components. In various embodiments, the exemplary tibial template can replace a significant number of surgical tools as well as reduce surgical times. The tibial template can be placed after predetermined cuts to the tibia and femur are performed. The tibial template is placed onto the resected surface of a tibia, and a femoral trial is placed onto the resected femur. The surgeon may subsequently begin to measure the balancing, rotation, and alignment of the knee joint components in flexion (90 degrees) and extension (0 degrees) for accurate placement of the tibial tray implant.

In use, the femoral trial implant condylar surfaces 360 will desirably rotate within and relative to the condylar receptacles 340, with improper movement or other motion mimicking the balancing, alignment and rotation of the knee joint components. The user handle 350 can be manipulated from various directions while the femoral trial implant is seated on the tibial template, to mimic rotation and/or repositioning of the tibial template to a desired position to achieve a desired motion of the implant. The coronal laxity—angles between the cut surfaces of the femur and tibia—can be measured in extension and in flexion. The surgeon may choose to impose a valgus or varus force just above the knee on the lateral or medial side, with the knee counter-supported at a selected angle of flexion to confirm proper laxity. During this time, the surgeon may measure the gaps using standard available tools in the operating room. Slightly greater laxity in the lateral than medial side may be acceptable because even normal knees have unbalanced soft-tissue tension. Greater laxity in the lateral side can be important in knee surgeries, because equal tension on both sides may impair smooth axial tibial rotation in flexion. The surgeon may decide to change the tibial template with increased or decreased thicknesses and varying dimensions if the gap measurements are not optimal in flexion and extension. Good soft-tissue balance in both extension and flexion can enable long-term stability after knee surgery. In various embodiments, the template may incorporate modular and/or removable inserts that alter the thickness and/or spacing of the medial and/or lateral sides (either both together, or each individually). If desired, such inserts may be added to the bottom of the template and/or to the respective medial and lateral faces of the template.

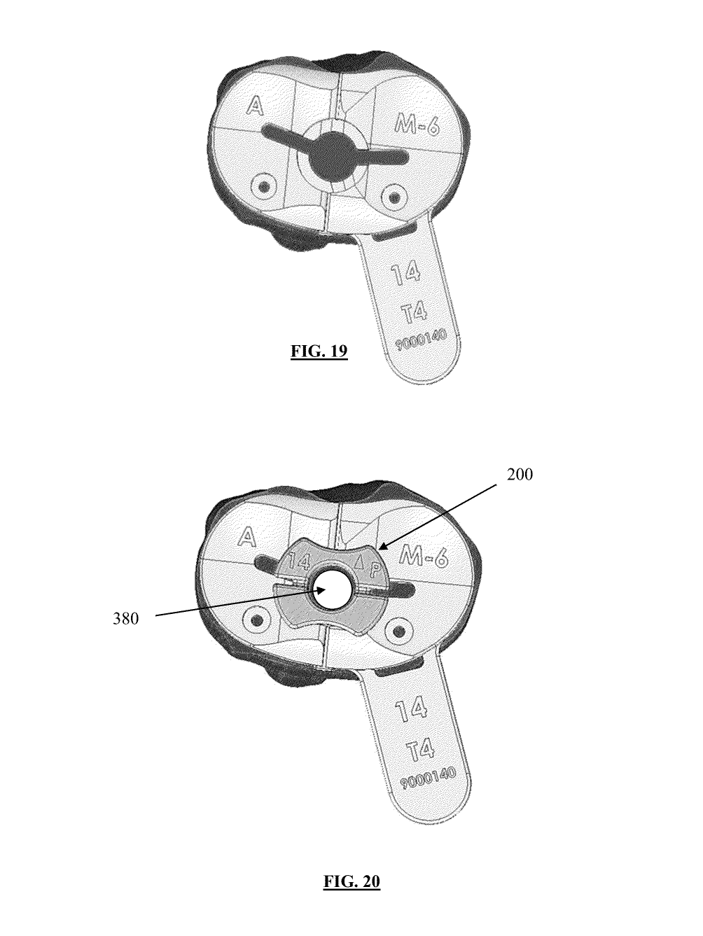

FIG. 18 depicts a tibial template and a tibia, highlighting the positioning pin holes. Once the proper positioning, balancing, and alignment has been achieved using the proper tibial template size, shape and orientation, the tibial template 20 can be fixed into position. The tibial template 20 can be fixed into position by drilling through the positioning holes 140 that are located in the medial and lateral condyle receptacles of the tibial template. The drill will be inserted into the positioning holes 140 and the surgeon will drill until reaching the counter bore as a positive stop, if desired. FIG. 19 depicts the top view of FIG. 18.

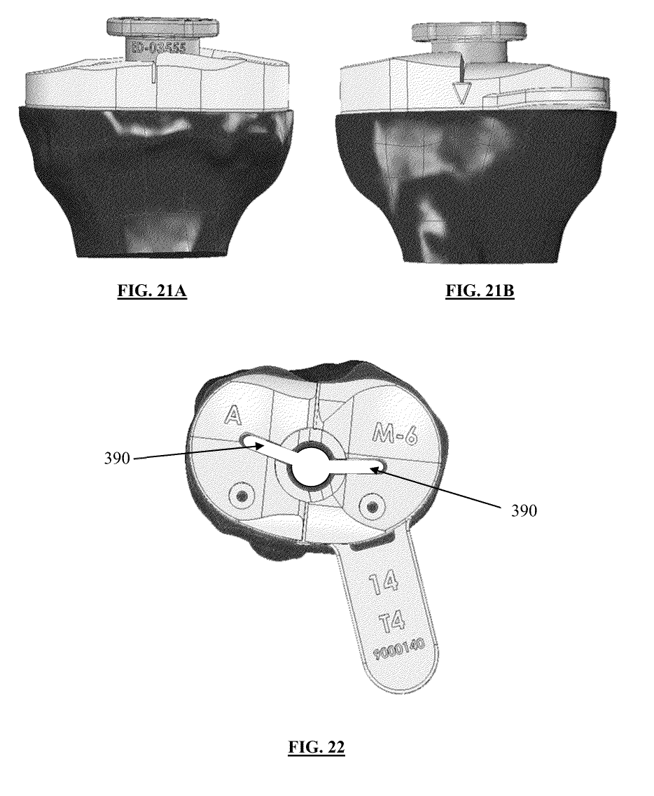

FIG. 20 depicts a top view of the template of FIG. 18, after the tibial template bushing 200 has been aligned with the tibial template in its unlocked position. The surgeon can rotate the tibial template bushing 200 a desired amount, such as 90 degrees, to have the alignment indicator align with the bushing indicator to engage the locked position. The surgeon may drill a bore through the intramedullary canal using standard operating tools during surgery. FIGS. 21A and 21B depict the posterior and anterior view of the tibial template and the tibial template bushing assembly.

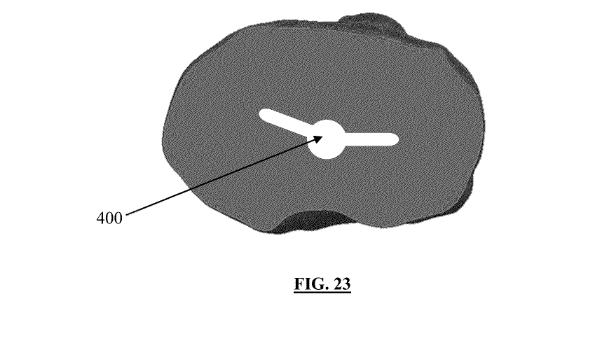

FIG. 22 depicts the top view of the tibial template highlighting the bore through the intramedullary canal after the bushing has been removed. The surgeon may keel punch the tibial surface, using a keel punch provided by the manufacturer. FIG. 23 depicts the top view of the resected tibia that highlights the bore through the intramedullary canal and the keel punched extensions. The tibia can then receive the tibial tray implant.

By allowing for a quick and convenient method of assessing a knee implant throughout an entirety of its range of motion early in the procedure and/or at virtually any point during the surgical procedure, the various embodiments disclosed herein can facilitate the proper balancing, rotation, laxity accommodation and implant component alignment during total knee surgery. The ability of the surgeon to assess such conditions quickly and accurately, and to alter the alignment of support structures and the implant components themselves, will have a direct and positive effect on the ultimate performance of the knee implant. In various embodiments, the methods and devices described herein can assist a surgeon to optimize implant function, valgus/varus alignment, joint rotation, patello-femoral tracking and optimal knee range of motion, which can be important factors contributing to a well-functioning knee implant and positive surgical outcomes.

What is claimed is:

1. A tibial template for use during treatment of a knee joint of a patient, the tibial template comprising:
a medial condylar receptacle surface and a lateral condylar receptacle surface, wherein at least a portion of the medial condylar receptacle surface and/or the lateral condylar receptacle surface has a shape based, at least in part, on patient-specific information;
a bottom surface generally opposite the receptacle surfaces;
a perimeter having a shape based, at least in part, on patient-specific information; and
an opening sized and shaped to accommodate a tool selected from the group of tools consisting of a cutting tool, a drilling tool, a keel punch, and combinations thereof.

2. The tibial template of claim 1, wherein the opening comprises a keel guide.

3. The tibial template of claim 1, wherein the opening comprises a drill guide hole.

4. The tibial template of claim 1, further comprising one or more position pin holes.

5. The tibial template of claim 1, further comprising a medial position pin hole passing through a portion of the medial condylar receptacle surface and a lateral position pin hole passing through a portion of the lateral condylar receptacle surface.

6. The tibial template of claim 1, wherein a shape of at least a portion of the medial condylar receptacle surface and/or the lateral condylar receptacle surface is based, at least in part, on a shape of at least a portion of a knee implant prosthesis.

7. The tibial template of claim 1, wherein a shape of at least a portion of the medial condylar receptacle surface and/or the lateral condylar receptacle surface is based, at least in part, on a shape of at least a portion of a trial knee prosthesis component.

8. The tibial template of claim 1, wherein a distance from the bottom surface to the medial condylar receptacle surface comprises a medial height and a distance from the bottom surface to the lateral condylar receptacle surface comprises a lateral height, and wherein the medial height and/or the lateral height is based, at least in part, on patient-specific information.

9. The tibial template of claim 8, wherein the medial height is different than the lateral height.

10. The tibial template of claim 1, wherein the shape of the perimeter substantially matches at least a portion of a perimeter of a resected tibia of the patient.

11. A system for treating a knee joint of a patient, the system comprising:
a femoral implant; and
a first tibial template, the first tibial template comprising:
a medial condylar receptacle surface and a lateral condylar receptacle surface, wherein at least a portion of the medial condylar receptacle surface and/or the lateral condylar receptacle surface has a shape based, at least in part, on patient-specific information;
a bottom surface generally opposite the receptacle surfaces;
a perimeter having a shape based, at least in part, on patient-specific information; and
an opening sized and shaped to accommodate a tool selected from the group of tools consisting of a cutting tool, a drilling tool, a keel punch, and combinations thereof.

12. The system of claim 11, further comprising a second tibial template, and wherein a portion of the first tibial template has a first height and a corresponding portion of the second tibial template has a second height, the second height being different than the first height.

13. The system of claim 12, further comprising a third tibial template, wherein a portion of the third tibial template corresponding to the portion of the first tibial template has a third height, and wherein the second height is larger than the first height and the third height is smaller than the first height.

14. The system of claim 11, further comprising a drill stop, the drill stop configured to be releasably secured within the opening of the first tibial template.

15. The system of claim 11, wherein a shape of at least a portion of the medial condylar receptacle surface and/or the lateral condylar receptacle surface is based, at least in part, on a shape of at least a portion of the femoral implant.

16. The system of claim 11, further comprising a femoral trial implant.

17. The system of claim 16, wherein a shape of at least a portion of the medial condylar receptacle surface and/or the lateral condylar receptacle surface is based, at least in part, on a shape of at least a portion of the femoral trial implant.

18. The system of claim 11, wherein the opening comprises a keel guide.

19. The system of claim 11, wherein a distance from the bottom surface to the medial condylar receptacle surface comprises a medial height and a distance from the bottom surface to the lateral condylar receptacle surface comprises a lateral height, and wherein the medial height and/or the lateral height is based, at least in part, on patient-specific information.

20. The system of claim 19, wherein the medial height is different than the lateral height.
